SEQUENCE LISTING (9 sequences)

SEQ ID NO: 1 - DNA, 36 bp, Artificial Sequence
  Oligonucleotide P53-U
gcagccgcgaagtatatctgcaactcatcctgcatg

SEQ ID NO: 2 - DNA, 36 bp, Artificial Sequence
  Oligonucleotide P53-L
catgcaggatgagttgcagatatacttcgcggctgc

SEQ ID NO: 3 - DNA, 36 bp, Artificial Sequence
  Oligonucleotide FLU-U
gcagccgcgggaatactagggttcgtatttacacta

SEQ ID NO: 4 - DNA, 36 bp, Artificial Sequence
  Oligonucleotide FLU-L
tagtgtaaatacgaaccctagtattcccgcggctgc

SEQ ID NO: 5 - DNA, 42 bp, Artificial Sequence
  Oligonucleotide TAT-U
ggccgctacggacgaaagaagaggcgtcaacgacgcagacca

SEQ ID NO: 6 - DNA, 42 bp, Artificial Sequence
  Oligonucleotide TAT-L
tggtctgcgtcgttgacgcctcttctttcgtccgtagcggcc

SEQ ID NO: 7 - protein, 9 aa, Artificial Sequence
  p53 CTL reactive peptide
KYICNSSCM

SEQ ID NO: 8 - protein, 9 aa, Artificial Sequence
  Influenza A matrix protein peptide reactive with influenza-specific CTLs
GILGFVFTL

SEQ ID NO: 9 - protein, 4 aa, Artificial Sequence
  A C-terminal sequence optionally fused to HLA class I-associating peptides
KDEL

(19) United States
(12) Patent Application Publication
(10) Pub. No.: US 2006/0099216 A1
(43) Pub. Date: May 11, 2006

(54) Peptide delivery

(75) Inventors: Donald Leonard Nicholas Cardy, Aston-le-Walls (GB); Frank Joseph Carr, Balmedie (GB)

Correspondence Address: KIRKPATRICK & LOCKHART NICHOLSON GRAHAM LLP;(FORMERLY KIRKPATRICK & LOCKHART LLP), 75 STATE STREET, BOSTON, MA, 02109-1808, US

(73) Assignee: Biovation Limited, Aberdeen (GB)

(21) Appl. No.: 11/238,179

(22) Filed: Sep. 27, 2005

Related U.S. Application Data
(63) Continuation of application No. 08/737,457, filed on Mar. 12, 1997, now abandoned.

(30) Foreign Application Priority Data
Aug. 31, 1994 (GB) 9417461
May 13, 1994 (GB) 9409643

(51) Int. Cl. A61K 39/00; C07K 14/47

(52) U.S. Cl. 424/184.1; 530/350

(57) ABSTRACT

Disclosed is a chimaeric polypeptide comprising: a binding portion having specific binding affinity for a eukaryotic target cell surface component and an effector portion comprising an amino acid sequence capable of exerting a biological effect; whereby binding of the polypeptide to the cell surface component induces internalisation of at least the effector portion so as to allow the amino acid sequence to exert its biological effect, together with a vaccine comprising the chimaeric polypeptide of the invention, and a method of modulating the immune response of a human or animal subject.

This application is a continuation of US application Ser. No. 08/737,457, which is the US national stage of international patent application PCT/GB95/01107, filed May 15, 1995, which claims the benefit of (i) United Kingdom patent application 9409643, filed May 13, 1994, and (ii) United Kingdom patent application 9417461, filed Aug. 31, 1994. The disclosures of each of the above-referenced patent applications are incorporated by reference herein in their entireties.

### FIELD OF THE INVENTION

This invention relates to chimaeric polypeptides designed to bind to a target cell, to vaccines comprising the chimaeric polypeptides, and to a method of modulating the immune response of a human or animal subject.

In particular, the invention relates to chimaeric polypeptides designed for the efficient delivery of one or more effector molecules to a target cell for subsequent internalisation by the cell. In specific embodiments the invention relates to recombinant antibody molecules containing immunodominant peptide sequences whereby these antibody molecules enter cells via internalising antigens or receptors and are subsequently processed to release peptides for antigen presentation leading to either induction or inhibition of an immune response. The invention also relates to methods for treatment and prevention of disease using these new types of recombinant antibodies or proteins and methods for generating these molecules.

### BACKGROUND OF THE INVENTION

The finding that T-cells recognise antigen as peptides bound to major histocompatability complex (MHC) molecules has given rise to attempts to use defined synthetic peptides corresponding to immunodominant T-cell epitopes to either activate or tolerise T-cells to specific antigens. Generally, the approach used has been to immunise with the immunogenic peptide in order to induce a T-cell response relying on the efficient processing and antigen presentation by professional immune system cells to induce a T-cell response comprising either a predominant response by CD8+ cytotoxic T lymphocytes (CTLs) for peptide presented in conjunction with MHC class I molecules or a predominant CD4+ helper T-cell response for peptide presented in conjunction with MHC class II molecules. For the treatment of cancer, there is now a major interest in the induction (or adoptive transfer) of CTLs specific for tumour-associated antigens such as the MAGE-1 antigen produced by a high proportion of human melanoma tumours (van der Bruggen et al., 1991 Science 254, 1643). There is also an interest in the identification of peptides associated with diseases such as autoimmunity in order to induce tolerance by administration of the autoimmune peptide and subsequent presentation and overstimulation leading to abrogation of peptide-specific CTLs (e.g. Aichele et al., 1994 PNAS 91, 444). There is additionally an interest in the identification of peptides associated with diseases such as allergy in order to redirect a TH2 response induced by the allergen to a TH1 response by immunising with a peptide homologue of the TH1-inducing epitope. Another approach is to immunise with altered peptides which, upon presentation, can bind to but not activate a specific T-cell thus antagonising deleterious responses of such T-cells.

The present inventors consider that there are three main limitations to the direct use of peptides for activation or tolerance of specific T-cells to antigens involved in disease-processes. Firstly, peptide-induced activation of cytotoxic T cells for the subsequent destruction of aberrant disease-associated target cells is restricted to target cells which present the specific peptide, usually professional (constitutive) or facultative antigen presenting cells and also cells expressing abnormal antigens such as cancer cells expressing the MAGE-1 antigen. Thus, it is currently not possible to direct the activation of T cells by antigen presentation by specifically chosen cell types. Secondly, peptide-induced activation or tolerance relies on the efficient uptake and presentation of peptide by professional or facultative antigen presenting cells; the efficiency of presentation is highly dependent on factors such as peptide concentration and peptide formulation with adjuvants which are variable for different peptides and do not always result in a specific T-cell response. Thirdly, peptide presentation is MHC-restricted and dependent on specific HLA type and thus the activation or tolerance of T cells to a certain immunodominant peptide is dependent on the HLA type of the individual. Moreover, exogenously added peptides (such as used in vaccines) will normally be taken up and processed by cells in such a way that they are presented to the immune system in association with MHC class II antigens only—exogenously added peptides tend not to enter the processing pathway which leads to association with MHC class I antigens. which association is essential for eliciting a CTL response. This problem has not so far been satisfactorily addressed.

### SUMMARY OF THE INVENTION

In a first aspect the invention provides a chimaeric polypeptide comprising a binding portion having specific binding affinity for a eukaryotic target cell surface component and an effector portion comprising an amino acid sequence capable of exerting a biological effect; whereby binding of the polypeptide to the cell surface component induces internalisation of at least the effector portion so as to allow the amino acid sequence to exert its biological effect.

Typically the whole of the chimaeric polypeptide will be internalised. but this is not essential.

Preferably the binding portion comprises an immunoglobulin molecule (e.g. an antibody) or an effective portion thereof. The term “effective portion” is intended to denote those portions of immunoglobulin molecules which retain some degree of specific binding affinity, and includes Fab, Fv, SCA and scFv fragments.

Desirably the biological effect mediated by the amino acid sequence will be an immunomodulatory effect. Preferably the amino acid sequence capable of exerting an immunomodulatory effect will be an immunodominant peptide, typically a T cell epitope. The effector portion may comprise a single amino acid sequence capable of exerting an immunomodulatory effect or may comprise a plurality of such sequences. Where the effector portion of the chimaeric polypeptide comprises a plurality of such amino acid sequences, they may be joined to form a single “domain”-like structure or may be disposed at different points in the polypeptide. Similarly, where the effector portion comprises a plurality of amino acid sequences, they may be repeats of the same sequence or may be single copies of various different amino acid sequences.

Conveniently the effector portion will also comprise a signal which directs the amino acid sequence to a specific cellular compartment. Such signals are further discussed below.

The invention also provides a vaccine comprising the chimaeric polypeptide defined above, and a method of regulating the immune response of a human or animal subject. The invention also includes within its scope a method of making the chimaeric polypeptides defined above.

Accordingly, instead of relying on individual peptides being taken up and presented by professional or facultative antigen-presenting cells, the present invention uses antibody or protein molecules specific for internalising antigens or receptors on any cell type to deliver one or more peptides internally to these cells. Upon digestion and processing of the recombinant antibody/protein-peptide molecules, the peptides can then be transported to and presented on the cell surface by appropriate MHC molecules or can interact with other intracellular molecules and remain within the cell. In particular, the present invention relates to a new class of antibody or protein molecule produced by recombinant DNA methods to include within the protein the sequence of one or more immunodominant peptides. Thus the invention provides, for the first time, a means of targeting antigen presentation to a particular chosen cell with the main requirement of an internalising antigen or receptor bound by an antibody or protein containing the peptide antigen to be presented (optimally in multiple copies).

Advantages of the present invention for the activation or tolerance of T-cells are several-fold. Firstly, by using an antibody or protein to deliver immunodominant peptides, these peptides can be delivered, via an internalising antigen, to cells which do not normally present such peptides. In the case of cancer cells for example, a cancer cell can then present one or more peptides designed to activate CTLs to subsequently destroy the cancer cell. In the case of normal cells subject to autoimmune destruction by CTLs, these peptides can be delivered via an internalising antigen or receptor either as high doses of normal immunodominant peptides to induce tolerance by abrogation of the usually destructive CTLs or as altered peptides which antagonise the disease-associated T-cell activation. Secondly, several different peptide sequences or multiple copies of the same peptide sequence can be associated with a single antibody or protein molecule, thus providing either the possibility for a choice of peptides for presentation by the appropriate MHC haplotype in the individual immunised or the possibility for efficiently delivering a high dose of peptide via multiple copies on the antibody molecule. Thirdly, by using an antibody or protein to deliver immunodominant peptides, these peptides can be efficiently delivered, via an internalising antigen, to specific professional antigen presenting cells as an alternative to the less specific endocytosis by these cells; specific antigen-presenting cells can thus be targeted in order to better control the type of immune response to the immunodominant peptide.

It will be understood that antibodies or proteins of the present invention can be used to deliver peptides into cells, either for subsequent binding to and presentation by MHC molecules, or for binding to specific intracellular molecules to produce effects such as blocking intracellular protein-protein interactions (such as those involved in intracellular signaling). It will be understood that the present invention can apply to both class-I and class-II MHC-restricted immunodominant peptides and can potentially be applied to the targeted induction of an immune response to many different antigens in many different disease states such as cancer (induction of CTLs by cancer cells), autoimmunity (antagonism of T-cell activation or induction of tolerance), infection (induction of CTLs by infected cells, antagonism of T helper cell activation by superantigen), allergy (diversion of a TH2 to a TH1 response by the immunodominant peptide) and inflammation (antagonism of T-cell activation). It will also be understood that antibodies or proteins produced by the present invention could include the so-called “universal” immunodominant peptides (e.g. tetanus toxin and influenza nucleoprotein peptides) against which a high proportion of individuals might already be sensitised to by vaccination or infection. It will also be understood that, for efficient presentation of MHC class I-associated peptides by antibodies or proteins produced by the present invention, the inclusion in the molecule of sequences facilitiating entry of these peptides into the cytoplasm from compartments such as the endosomes or trans golgi network may be required and that certain such sequences (or domains) already known in the literature (such as the translocation domains of toxins such as Pseudomonas exotoxin (Donnelly et al., 1993 PNAS 90, 3530), the HIV-1 tat protein (Fawell et al. 1994 PNAS 91, 664) or the endosome-disrupting functions of viruses such as adenovirus (Curiel et al., 1991 PNAS 88, 8850). It will also be understood that antibodies or proteins produced by the present invention could include a mixture of different peptides which associate with different MHC class I or class II molecules thus providing for a broader prospect of inducing an immune response in different population groups carrying different HLA types. It will also be understood that the antibodies or proteins of the present invention could be used in several different ways in disease treatment and prevention including to deliver peptides ex vivo for the induction of CTLs prior to adoptive immunotherapy and to enhance the T-cell activating, blocking or abrogating ability of cells already presenting the specific peptide(s) or analogues thereof. It will be understood that antibodies or proteins of the present invention could be targeted to professional antigen presenting cells such as. for example, macrophages (e.g. via the FcRI receptor) and B cells (e.g. via surface immunoglobulin molecules). Finally, it will be understood that the antibodies or proteins of the present invention might be used for disease treatment or prevention either alone or in combination with other molecules which potentiate the interaction with T-cells such as molecules which up-regulate class I and class II MHC expression in certain cell types such as interferon-gamma or that the antibodies or proteins of the present invention might themselves be used as vaccines through the inclusion of adjacent immunodominant B and T-cell epitopes optimally as multiple copies and targeted to professional antigen presenting cells.

It will be understood by those skilled in the art that a range of internalising antigens might be targeted by the polypeptides of the invention in delivering peptides intracellularly to specific cells. For example, MHC class I or class II molecules might be targeted on professional antigen-presenting cells in order, for example, to promote a strong immune response against the delivered peptides and to deliver the peptides to these cells without use of an adjuvant. In addition, other professional antigen-presenting cells might be targeted such as mucosal cells and dendritic cells and also specific lymphocyte subsets might be targeted through the use of antibodies which bind to lymphocyte cell surface internalising antigens such as surface immunoglobulins (sIg).

It will be understood by those skilled in the art that immunodominant peptides might be included at many different locations in an antibody molecule using recombinant DNA. It will be understood that incorporation or conjugation of these peptides should be such that, as required. antibody function such as binding affinity and rate of clearance is not impaired. Alternatively, it will be understood that peptides could be incorporated as part of an incomplete recombinant antibody fragment such as an Fab, an Fv or an SCA (single chain-antibody) fragment. Depending on the application. it might be preferable to include the immunodominant peptides in a protein domain or structure designed to either mask the peptides from specific recognition by circulating or cell-bound molecules or to expose the peptides such that some immune recognition is possible instead of direct uptake by an internalising antigen. For peptides which particularly stimulate a good B-cell response following internalisation and presentation (particularly MHC class-II restricted), it might be preferable to mask the peptides from specific recognition by circulating antibodies in order to avoid premature clearance by the immune system. Finally, it will be understood that a bivalent (or multivalent) antibody might be used consisting of one part binding to the internalising antigen and another part with no specific binding specificity but including the immunodominant peptide either within or attached to the antibody structure (including immunodominant peptides insertion at the usual sites for CDRs (complementarity-determini-ng regions).

### BRIEF DESCRIPTION OF THE FIGURES

The invention will now be further described by way of illustration and with reference to the accompanying drawings, wherein FIGS. 1 to 9 illustrate a number of possible designs for antibodies including immunodominant peptides either in discrete multi-peptide domains (FIGS. 1 to 8) or as individual peptide sequences included to replace CDRs (FIG. 9). These Figures should not be considered an exclusive set of designs for antibodies containing immunodominant peptides and should not be considered as limiting the scope of the invention. For these Figures, antibodies are drawn comprising CDRs, VH and VL domains i.e. variable region heavy and light chain domains (including the CDRs), CL domains i.e. light chain constant domains, and CH1-3 (i.e. heavy region constant domains). The portions of the molecule comprising the amino acid sequences capable of exerting a biological effect are shown dotted.

FIG. 1 illustrates an antibody whereby the immunodominant peptide domain including multiple copies of the same immunodominant peptide or, alternatively, individual copies of multiple peptides (for example, to compensate for MHC-restriction by providing a range of options covering different MHC haplotypes) has been fused adjacent to the hinge (“H”) region in the heavy chains upstream from the CH2 domains;

FIG. 2 illustrates an antibody whereby the immunodominant peptide domain has been fused adjacent to the hinge region in the heavy chains thus replacing the CH2 and CH3 domains to create a Fab-peptide hybrid antibody;

FIG. 3 this illustrates an antibody whereby the immunodominant peptide domain has been fused adjacent to the hinge region in the heavy chains replacing the CH2 domains;

FIG. 4 illustrates a SCA (single-chain antibody) whereby the immunodominant peptide domain has been fused adjacent to a recombinant Fv via a linker arm;

FIG. 5 illustrates an antibody whereby the CH3 domain has been replaced by the immunodominant peptide domain;

FIG. 6 illustrates an antibody whereby the immunodominant peptide domain has been fused adjacent to the VL region in the light chains thus replacing the CL domains:

FIG. 7 illustrates an antibody whereby the immunodominant peptide domain has been fused adjacent between the VH and CH1 domains in the heavy chains and between the VL and CL domains in the light chains;

FIG. 8 illustrates a bispecific antibody created by either the coexpression or thiol reduction/oxidation of a mixture of antibody heavy chains whereby in one the immunodominant peptide domain has been fused adjacent to the hinge region thus replacing the CH2 and CH3 domains;

FIG. 9 illustrates an antibody whereby 6 copies (or 6 variants) of the immunodominant peptide have been inserted in the VH/VL regions to replace the CDRs; and

FIG. 10 illustrates the typical organisation of an immunodominant peptide domain for HLA class I-associating peptides whereby several peptide copies arranged in a contiguous fashion are fused to a translocating domain (D) and optionally a C-terminal KDEL (SEQ ID NO:9)-like sequence.

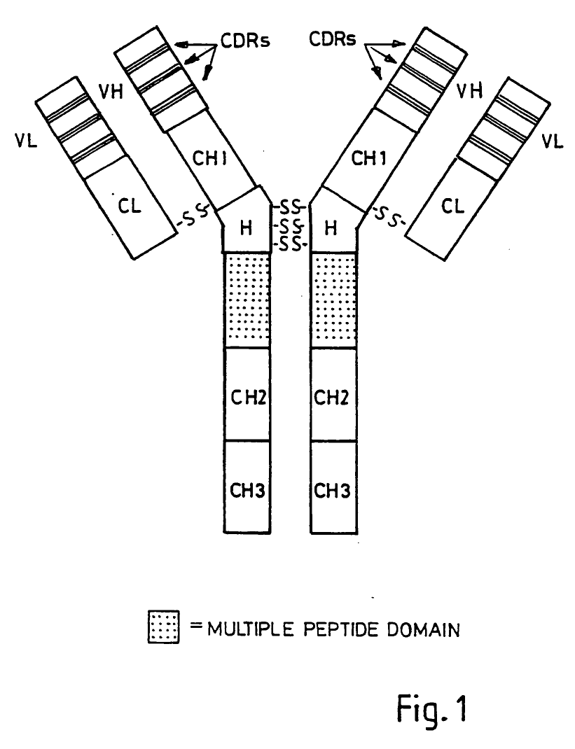

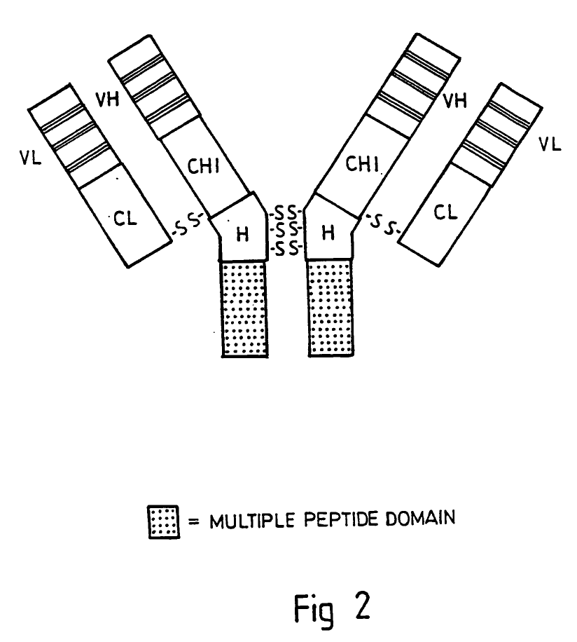

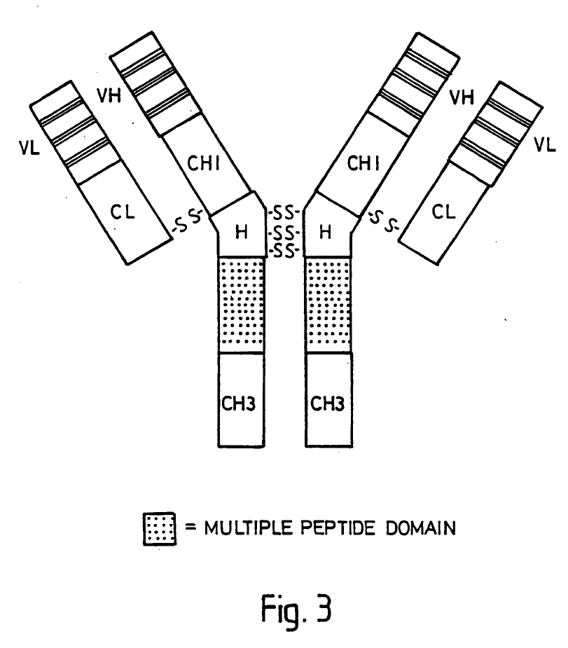

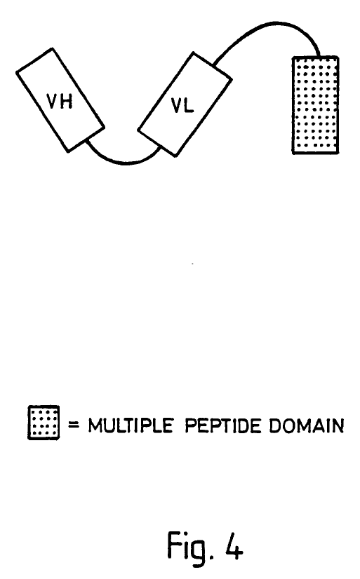

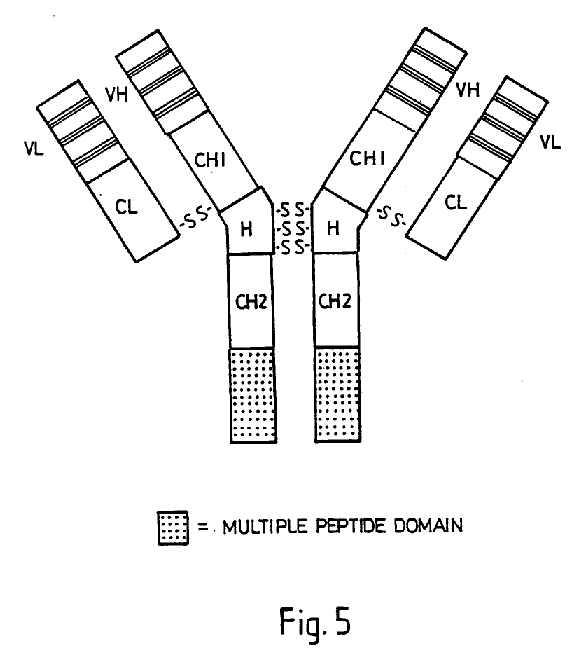

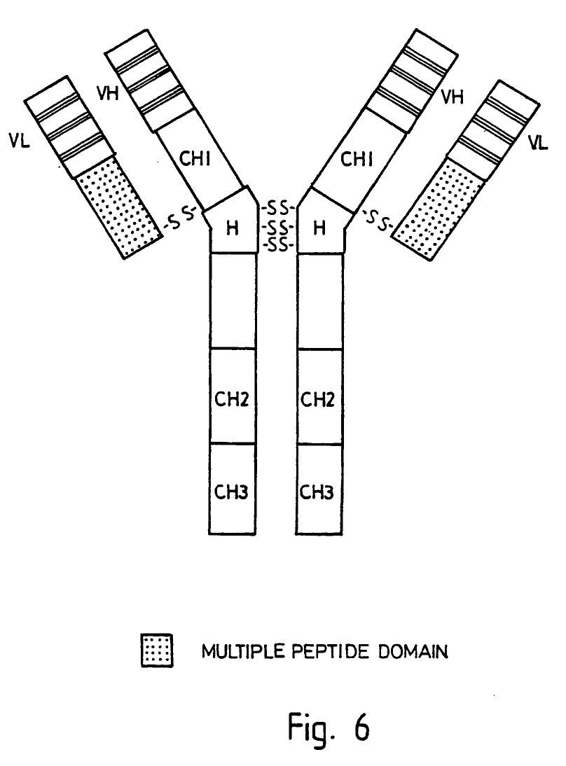

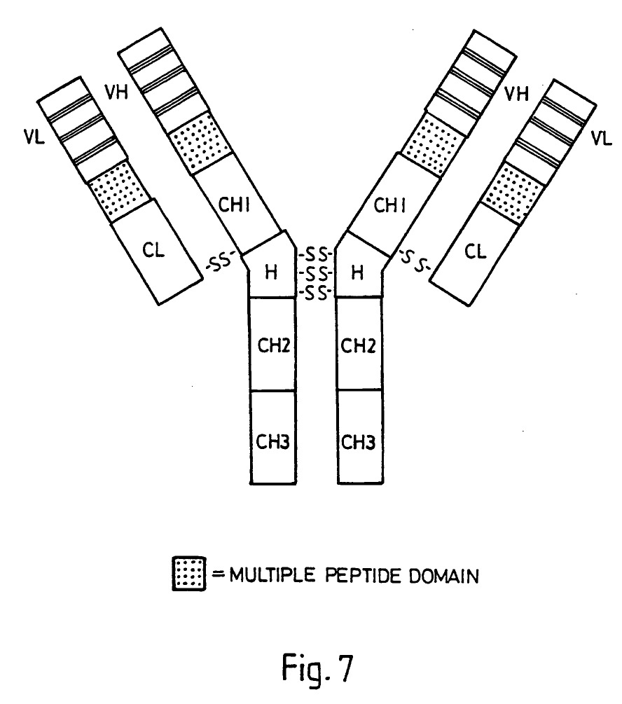

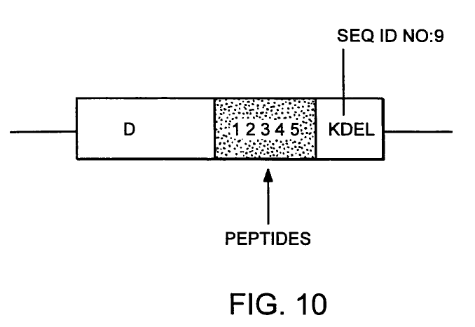

The peptide domains referred to in FIGS. 1 to 8 and depicted in FIG. 10 above could comprise either multiple repeats of the same immunodominant peptide or a range of different immunodominant peptide sequences within the same domain. In each case, the peptide sequences may be repeated in head-to-tail or head-to-head organisation, preferably with suitable amino acid spacing between the sequence repeats provided by non-immunodominant sequences. In each case, the immunodominant peptide content of heavy and light chains might be the same or different and peptide domains may be added to all the chains, or to the two heavy or the two light chains only, or to just one of the heavy and/or light chains or as part of a bispecific (or multispecific) antibody whereby the peptide domains might only be derived from one or more of the recombinant chains used to create the bispecific antibody. It will be apparent that the arrangement of immunodominant peptide sequences in the peptide domain might require optimisation for different peptides included in the domain and might need to be optimised in order to ensure efficient production of the recombinant antibody without disruption of the antibody-like structure, and that such optimisation should be a matter of comparatively simple trial and error for those skilled in the art. It will also be understood that optimisation of antibodies for effective processing of the immunodominant peptide domains might also be required. In each case, optimisation might require different arrangements of peptides in conjunction with each other, different flanking amino acid sequences between the immunodominant peptides (including introduction of sequences, such as acid-labile or peptidase sites, to promote cleavage upon internal processing outside the immunodominant peptide regions) and different combinations with other peptide or protein structures as required for various reasons (e.g. keeping the total molecule from misfolding or to keep the immunodominant peptides either internalised or externalised in a globular protein structure). It will also be apparent that, for HLA class I-associating peptides, the addition of a translocating domain (or domains) adjacent to the tandemly-arranged peptides might be required for efficient transport to, and processing in, the cytoplasm.

The following examples serve to illustrate the possible applications of antibodies of the present invention and should not be considered as limiting the scope of the invention.

### EXAMPLE 1

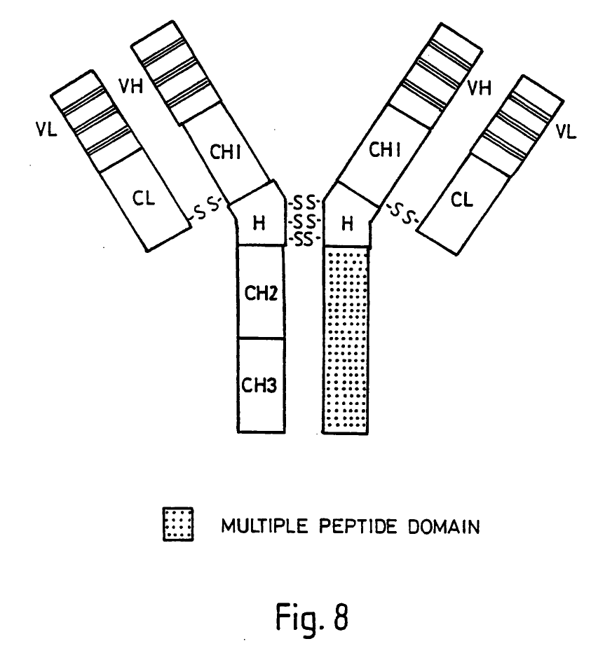

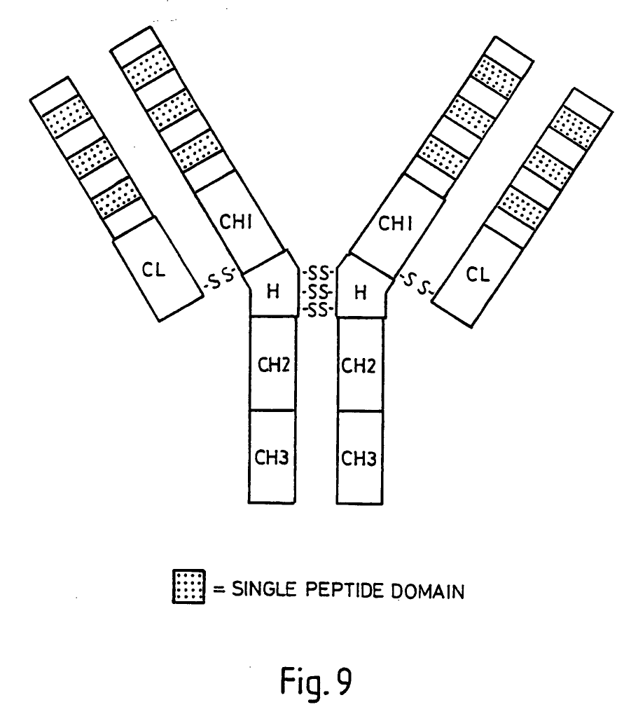

The generation of recombinant monoclonal antibodies suitable for use in treatment and prevention of human disease has been comprehensively described in the literature (for example: Boulianne et al., 1984 Nature 312, 643; Morrison et al., 1984 PNAS 81, 6851; Reichmann et al. 1988 Nature 332, 323; Queen et al., 1989 PNAS, p 10029) and methods have been developed for production of whole antibodies from mammalian and other eucaryotic cells and also for production of antibody fragments (e.g. Fv, Fab, and SCA fragments) from E. coli. For generation of antibodies from mammalian cells, all of the antibody designs depicted in FIGS. 1 to 3 and 5 to 9 can be produced using prior art methods including one or more cloning steps to insert sequences encoding peptides for delivery into cells within the antibody molecule. Methods for generation of bispecific antibodies as depicted in FIG. 8 are also known in the art for mammalian cells and bispecific antibody fragments can also be generated from bacteria. By way of example and to illustrate the utility of such antibodies for vaccination, a recombinant antibody was generated which was specific for the invariant chain of the MHC class II antigen on professional antigen-presenting cells. The starting point was the hybridoma cell line DA6.231 obtained from the European Collection of Animal Cell Cultures (ECACC No. 90110606). Cells were grown in RPMI1640 and 10% foetal bovine serum. mRNA isolation and the cloning of scFvs (single-chain Fvs) was by the protocol recommended in a “Recombinant Phage Antibody System” (Pharmacia, Milton Keynes, UK) using reagents in a kit supplied by the manufacturer with cloning into the pCANTAB5E cloning vector and K07 helper phage (both included in the kit). For isolation of specific scFvs, MHC class II antigen was prepared according to van Hevningen et al. (1982 Immunogenetics 16, 459-469) using the DA6.231 antibody for purification of MHC class II antigen from DAUDI lymphoblastoma cells (ECACC No. 85011437). The soluble antigen preparation derived from 10.sup.8 cells was absorbed on 3.times.T25 tissue culture flasks (Costar) for subsequent panning of phage particles and recovery of residual binding phage particles after three panning steps. Clones of recovered phage were checked for specific binding to MHC class II antigen by subsequent ELISA assays using class II antigen or irrelevant antigens absorbed into 96-well plastic microtitre plates. Peroxidase-labeled anti-M13 antibody was used for analysis as supplied in the Pharmacia kit. An individual phage antibody, termed anti-MHCII, was finally used to infect HB2151 cells to produce soluble recombinant phage antibodies.

All other DNA manipulation steps were performed according to Sambrook et al. (Molecular Cloning, A Laboratory Manual, Cold Spring Harbor Laboratory, Cold Spring Harbor, second edition, 1989). For the insertion of peptide sequences, synthetic oligonucleotides were synthesised using an Applied Biosystems 430A synthesiser and purified by reverse phase HPLC. Sequences were as follows;

(SEQ ID NO: 1)

P53-U:
5′-GCAGCCGCGAAGTATATCTGCAACTCATCCTGCATG-3′

(SEQ ID NO: 2)

P53-L:
3′-CGTCGGCGCTTCATATAGACGTTGAGTAGGACGTAC-5′

(SEQ ID NO: 3)

FLU-U:
5′-GCAGCCGCGGGAATACTAGGGTTCGTATTTACACTA-3′

(SEQ ID NO: 4)

FLU-L:
3′-CGTCGGCGCCCTTATGATCCCAAGCATAAATGT- GAT-5′

(SEQ ID NO: 5)

TAT-U:
5′-GGCCGCTACGGACGAAAGAAGAGGCGT- CAACGACGCAGACCA-3′

(SEQ ID NO: 6)

TAT-L;
3′-CCGGCGATGCCTGCTTTCTTCTCCGCAGTTGCTGCGTCTGGT-5′

The oligonucleotides P53-U and -L (encoding the p53 CTL reactive peptide KYICNSSCM SEQ ID NO: 7 (Noguchi et al., 1994 Proc. Natl. Acad. Sci. USA 91, 3171-3175) and FLU-U and -L (encoding the influenza A matrix protein peptide GILGFVFTL SEQ ID NO: 8 reactive with influenza-specific CTLs (Gammon et al., 1992 J Immunol. 148, 7-12) were 5′-labelled with .sup.32P using polynucleotide kinase and .sup.32P ATP and annealed together, self-ligated at 37.degree. C. for 4 hours using T4 DNA ligase (Life Technologies, Paisley UK) and analysed on a preparative polyacrylamide sequencing gel. The bands at 180 base pairs representing 5 self-ligated copies of P53-U/L or FLU-U/L were purified, ligated to phosphorylated NotI linkers (#1127, New England Biolabs, Hitchin, UK) and digested with NotI (Pharmacia).

pCANTAB5 anti-MHCII scFv phage DNA was digested with NotI (Pharmacia). To some samples, TAT-U and TAT-L oligonucleotides were annealed and ligated directly with the NotI-digested vector and used to transform E. coli TG1 cells. DNA from recombinants carrying the TAT sequences was further digested with NotI (anti-MHCII/tat) for subsequent cloning of P53 and FLU pentamers. NotI digested anti-MHCII or anti-MHCII/tat was ligated with the purified 180 base pair bands of p53 or flu epitopes and used to transform TG1 cells. Following cloning and checking for binding to MHC class II antigen by ELISA, soluble antibodies were generated by phage infection of HB2151 cells according to instructions with the Recombinant Phage Antibody System.

For preparation of cytotoxic T lymphocytes (CTLs) specific for the p53-derived peptide as above, the in vivo mouse peptide immunisation and sensitisation procedure of Noguchi et al. (loc. cit.) was followed to produce long-term CTL lines. For testing of antibodies for ability to induce CTL activity, target mouse Sp2/0 cells (ATCC CRL-1581) were used and maintained in DMEM and 10% foetal bovine serum. For CTL assays, cells at 5×10⁵ cells/ml were labeled overnight with 20 μCi (7.4 MBq). ⁵¹Cr chromate. Cells were then pelleted. washed in medium and resuspended at 5×10⁵ cells/ml in medium plus dilutions of the antibody fragments or 10 μg/ml peptide KYICNSSCM SEQ ID NO: 7 (“p53 peptide only”) for 4 hours at 37° C. Cells were then repelleted, washed twice in PBS (phosphate-buffered saline) and plated at 5×10³ cells in 100 μl in RPMI 1640 medium plus 10% foetal bovine serum in 24-well plates. 100 μl of CTLs were then added to give effector:target ratios of 20:1, 10:1 and 5:1 and incubated for 4 hours at 37° C. After incubation, 100 μl of culture supernatant was carefully removed from each well into an eppendorf tube, centrifuged and triplicate 20 μl aliquots of supernatant were counted in a scintillation counter. Percent specific release was calculated as [(release by effector cells-spontaneous release)/(maximum release-spontaneous release)]×100. Results were as follows;

| | % Specific ⁵¹Cr | | |
|---|---|---|---|
| | Release at E/T Ratio | | |
| Treatment | 20:1 | 10:1 | 5:1 |
| Anti MHCII-P53 | 23 | 23 | 17 |
| Anti MHCII-P53/tat | 47 | 43 | 27 |
| Anti MHCII-FLU/tat | 6 | 4 | 2 |
| Anti MHCII (no peptides) | 7 | 5 | 2 |
| p53 peptide only | 46 | 57 | 41 |

These results demonstrated the induction of cell lysis following incubation with antibodies specific for MHC class II molecules and carrying the p53 peptide sequence but not to anti-MHC class II carrying the influenza nucleoprotein peptide sequence. The results also showed an increased efficiency of lysis as a result of adding the tat sequence.

### EXAMPLE 2

To illustrate the utility of recombinant antibodies to direct CTLs to cancer cells, a recombinant antibody was generated which was specific for the MBr1 antigen (Menard et al., 1983 Cancer Research 43, 1295-1300) on certain cancer cells. The sequence of the immunoglobulin variable regions for the mouse antibody (Orlandi et al., 1989 Proc. Natl. Acad. Sci. USA 86, 3833-3837) was used as a starting point and was generated using standard methods by site-directed mutagenesis of the variable regions of plasmids M13-VHPCR1 and M13-VKPCR1 (Orlandi et al., loc. cit.). The MBr1 M13-VHPCR1 and M13-VKPCR1 plasmids were digested with PstI/BstEII and PvuII/BcII respectively and the small fragments were gel purified. These fragments were then PCR amplified using the Pharmacia Phage Antibody System and individual phage clones were screened by ELISA for binding to MCF7 breast cancer cells (ECACC No. 86012803) using the anti-M13 antibody. Soluble scFvs were prepared from positive phage by infection of HB2151 cells. Subsequently, P53 or FLU pentameric sequences were cloned into MBr1-specific phage with or without the tat peptide as in example 1 above.

Human cytotoxic T lymphocyte (CTLs) specific for the flu peptide GILGFVFTL SEQ ID NO: 8 were obtained from a normal HLA-A2 donor and were maintained as described by Bednarea et al., (1991 J. Immunological Methods 139, 41-47). Testing of antibodies for ability to induce CTL activity against target MCF7 cells was as for example 1 with effector:target ratios of 40:1, 20:1 and 10:1. Results were as follows;

| | % Specific ⁵¹Cr | | |
|---|---|---|---|
| | Release at E/T Ratio | | |
| Treatment | 40:1 | 20:1 | 10:1 |
| Anti MBr1-FLU | 13 | 6 | 5 |
| Anti MBr1-FLU/tat | 37 | 20 | 13 |
| Anti MBr1-p53/tat | 10 | 7 | 6 |
| Anti MBr1 (no peptides) | 8 | 7 | 6 |
| flu peptide only | 68 | 45 | 26 |

These results demonstrated the induction of cell lysis following incubation with antibodies specific for MBr1 molecules and carrying the flu and tat peptide sequences but not antibodies carrying the p53 peptide sequence. Omission of the tat sequence gave only a marginal increase in cell lysis by the scFv with the flu peptides.

### EXAMPLE 3

In another example to illustrate the invention, the antibody of FIG. 1 could be generated to include tandem-repeated multiple copies of the CTL-inducing nonapeptides derived from the genes, MAGE-1, 2 and 3 (van den Eynde et al., 1989 Int. J. Cancer 44, 643) with suitable spacing provided by short runs of non-immunodominant amino acids between the immunodominant peptides and with adjacently provided translocating domains such as from the HIV-1 tat protein. This could then be administered to a patient types as HLA-A1 harbouring a cancer with an internalising antigen (such as Lewis-Y) such that the antibody will internalise into the tumour and will potentially present the MAGE nonapeptides to the immune system. This may then activate MAGE-specific CTLs which will then lyse the tumour; alteratively, the patient could be preimmunised with MAGE peptides or an antibody such as in example 3 in order to expand and preactivate MAGE-specific CTLs.

### EXAMPLE 4

As an alternative to example 1, antibodies could be generated in the same manner but containing class-II MHC restricted peptides such as the tetanus toxin peptide P2 (Panina-Bordignon et al., 1989 Eur. J. Immunol. 19, 2237) and excluding the cytoplasm translocating domain. Such antibodies would be administered to a cancer patient with an appropriate HLA-DR type for presentation of P2. If the antibody is again specific for an internalising antigen on the cancer cells, then the antibody will internalise into the tumour and will potentially present the P2 peptides to the immune system. This may then activate P2-specific T helper cells to release lymphokines and to activate B cells and also may activate CTLs which will then lyse the tumour; in addition, a greater antitumour response may be generated if the patient is preimmunised with P2 peptide or an antibody such as in example 3 in order to expand and preactivate P2-specific CTLs.

### EXAMPLE 5

As alternatives to examples 1 and 2, antibodies could be generated in the same manner but with specificity for an internalising antigen on professional antigen-presenting cells, such as specificity for MHC class II antigen or specificity for surface-Ig on B cells, and including peptides presented by, for example cancer cells (e.g. for MAGE 1-3, a high proportion of human malignant melanoma cancers). If administered to a cancer patient with an appropriate MHC type for presentation of the peptides carried within the antibody, these should be processed and presented to the immune system by the antigen-presenting cells which would then stimulate an immune response against the cancer antigen represented by the peptides presented. In addition, antibodies from examples 1 and 2 might be used in combination with the type of antibody of this example in order to efficiently present the antigen on the target cancer cells for effective activation of T cells.

### EXAMPLE 6

As alternatives to examples 1 to 3, antibodies could be generated in the same manner for use in autoimmune diseases where the autoimmune immunodominant peptides are known. For example, T cells involved in the deterioration of normal tissue in the disease could be deleted or inhibited by creating an antibody specific for an internalising antigen on a professional antibody producing cell as in example 3 or by targeting the cells of the tissues subject to deterioration (e.g. thyroid cells via the TSH receptor for Graves Thyroiditis). In each case, the antibody would include multiple copies of the autoimmune immunodominant peptide to induce tolerance or would include altered peptides which, upon presentation, can bind to but not activate the deleterious T-cell thus inhibiting autoimmunity.

### EXAMPLE 7

As an alternative to example 3, antibodies could be generated with specificity for an internalising antigen on professional antigen-presenting cells, such as specificity for MHC class II antigen or specificity for surface-Ig on B cells, and including immunodominant peptides derived from infectious disease agents (e.g. the HIV V3-loop epitope conserved in a high proportion of HIV isolates). If administered to an infected patient with an appropriate MHC type for presentation of the peptides carried within the antibody, these should be processed and presented to the immune system by the antigen-presenting cells which would then stimulate an immune response against the infectious agent. Indeed, antibodies used in this manner might constitute effective vaccine agents as an alternative to vaccines comprising peptides or live/attenuated infectious agents which might not be efficiently processed by antigen-presenting cells.

### EXAMPLE 8

As an alternative to example 3, antibodies could be generated with specificity for an internalising antigen on professional antigen-presenting cells and including immunodominant peptides derived from allergens which normally induce a TH2 allergenic response. If administered preferably to the mucosal surfaces of a sensitised patient with an appropriate MHC type for presentation of the peptides carried within the antibody, these should be processed and presented to the immune system by the antigen-presenting cells which would then stimulate the induction of TH1 cells in preference to TH2 cells thus alleviating the allergenic response to the allergen. Indeed, antibodies used in this manner might constitute effective vaccine agents as an alternative to vaccinating with allergen preparations which might not be efficiently processed by antigen-presenting cells.

### EXAMPLE 9

As an alternative to example 3, antibodies could be generated with specificity for an internalising antigen on professional antigen-presenting cells and including immunodominant peptides derived from autoimmune antigens which normally induce a T helper response. If administered to an afflicted patient with an appropriate MHC type for presentation of the peptides carried within the antibody, these should be processed and presented to the immune system by the antigen-presenting cells which would then induce tolerance in the T helper clones responsible for the autoimmune response. Indeed, antibodies used in this manner might constitute effective alternatives to peptide vaccines.

1. A chimaeric polypeptide comprising: a binding portion having specific binding affinity for a eukaryotic target cell surface component and an effector portion comprising an amino acid sequence capable of exerting a biological effect; whereby binding of the polypeptide to the cell surface component induces internalisation of at least the effector portion so as to allow the amino acid sequence to exert its biological effect.

2-22. (canceled)
